(19) United States
(12) Patent Application Publication
(10) Pub. No.: US 2024/0240261 A1
(43) Pub. Date: Jul. 18, 2024

(54) FOCAL ADHESION KINASE SPLICING ISOMER AND APPLICATION THEREOF

(71) Applicant: CANCER HOSPITAL CHINESE ACADEMY OF MEDICAL SCIENCES, Beijing (CN)

(72) Inventors: Guangbiao ZHOU, Beijing (CN); Guizhen WANG, Beijing (CN); Dawei XIE, Beijing (CN); Beibei SUN, Beijing (CN); Zheng WANG, Beijing (CN)

(21) Appl. No.: 18/575,595

(22) PCT Filed: Mar. 17, 2022
(86) PCT No.: PCT/CN2022/081428

§ 371 Date: Apr. 4, 2024

(30) Foreign Application Priority Data
Jul. 1, 2021 (CN) 202110742821.2

(51) Int. Cl. C12Q 1/6886; G01N 33/573; G01N 33/574

(52) U.S. Cl. CPC C12Q 1/6886; G01N 33/573; G01N 33/57423; G01N 33/57484; C12Q 2600/136; C12Q 2600/158; G01N 2333/912

(57) ABSTRACT

The present disclosure provides focal adhesion kinase (FAK) splicing variants and use thereof, specifically, the use of reagents for detecting FAK splicing variants in the manufacture of diagnostic agents for diagnosis of small cell lung cancer (SCLC), and also the use of FAK splicing variants as a target in the screening and/or manufacture of a medicament for treatment of SCLC. The FAK splicing variants can be used as clinical diagnostic markers of SCLC, and are promising in clinical applications of FAK kinase inhibitors.

### TECHNICAL FIELD

The present disclosure relates to the technical field of splicing variants of focal adhesion kinase (FAK) and use thereof, and specifically to a method for detecting splicing variants of focal adhesion kinase in diagnosis of small-cell lung cancer and novel use thereof as a target of anti-cancer drugs, belonging to the field of medicine.

### BACKGROUND

Globally, there are about 2.1 million new cases of lung cancer and about 1.8 million deaths from lung cancer per year. Lung cancer has the highest incidence and mortality among all the cancer types. According to the tissue type, lung cancer is mainly divided into two classes: non-small cell lung cancer (NSCLC) and small-cell lung cancer (SCLC). SCLC is a class of poorly differentiated high-grade neuroendocrine malignant tumors, accounting for approximately 13% of all newly diagnosed lung cancers. Etiologically, 97% or more of SCLC can be attributed to smoking, and smoking cessation reduces the risk of SCLC. Under long-term stimulation by high levels of tobacco-derived carcinogens, the lung has a genome becoming instable, often putting patients under a high genetic mutational load. SCLC is generally a centralized lung cancer, metastasizes at an early stage, progresses rapidly, is sensitive to chemotherapy but shows recurrence in a short time and makes the therapy fail, and has a very poor prognosis, with a 5-year survival of less than 6%. For patients with limited-stage SCLC (tumor limited to a tolerable radiation field, seen in about ⅓ SCLC patients), the treatment is generally chemotherapy combined with synchronous thoracic radiotherapy. For patients with complete remission, prophylactic cranial irradiation (PCI) is also feasible. For patients with extensive-stage SCLC, the first-line standard therapy is platinum-based chemotherapy. Recently, it has been reported that the immune checkpoint inhibitor programmed death-ligand 1 (PD-L1) monoclonal antibodies (Atezolizumab and Durvalumab) combined with chemotherapy show efficacy on SCLC, but improve the survival of patients by only 2 to 2.7 months. Therefore, breakthroughs in the pathogenesis of SCLC are urgently needed to facilitate discovery of new driver genes, therapeutic targets, and molecular typing markers.

Focal adhesion kinase (FAK) is a non-receptor tyrosine kinase that has received considerable attention in recent years. Abnormal activation of FAK is positively correlated to the growth and the invasive and metastatic ability of many malignant tumors. High expression of FAK can be used as a marker for tumor prognosis. Abnormal activation of the FAK gene is mainly associated with a copy number increase, structural variations and mutations. An increase in FAK gene copy number has been reported in tumors of various tissue origins, such as lung, breast, colon, prostate and liver cancers of epithelial tissue origin, and myoma and gliomas of mesenchymal tissue origin, etc. An increase in FAK gene copy number is positively correlated with the growth and the invasive ability of various malignant tumors. Mutations of FAK gene detected in lung cancer mainly include E982K, P648S, H79Y, K259N, D918G, S517I, and T650A, but the function of the FAK gene mutations and their relationship with tumor development, especially lung cancer development, have not been reported yet. The main variants of human FAK identified so far are FAK^{del33}, FAK^{del26}, FAK^{+6.7} (box6,7, PWR) and FAK^{6.7} (box6,7)/FAK⁶ (box6)/FAK^{6.28} (box6,28)/FAK⁷(box7). FAK^{del33} has 1025 amino acids, and lacks exon No. 33 (amino acids 956-982), resulting in the failure of FAK to bind Integrins via Paxillin. FAK^{del26} has 982 amino acids and lacks exon No. 26 (amino acids 744-789), resulting in deletion of caspase-3/caspase-7-like cleavage site at the carboxyl terminus of FAK, thereby improving the stability and anti-apoptotic ability of FAK. FAK^{+6.7} and FAK^{6.7}/FAK⁶/FAK^{6.28}/FAK⁷ have inserted PWR, box6 (DEISGD), box28 (GINHCOHKVKKARRFLPLVFCSLEPPPT) and box7 (KSYGIDE) near the Tyr-397 autophosphorylation site, respectively, accompanied by the mutation of Thr to Ala, which removes the blocking by the N-terminal FERM domain and fully exposes the Tyr-397 autophosphorylation site, leading to enhanced FAK autophosphorylation activity. The relationship between structural variants of FAK and development and progression of tumors has been under research. In 2014, Fang et al. reported that, in breast and thyroid cancers, the expression of FAK^{del33} was closely related to tumor invasion, metastasis and prognosis. Similarly, FAK^{del26} was reported in breast cancer. FAK^{+6.7} and FAK^{6.7}/FAK⁶/FAK^{6.28} were reported in the nervous system. The present inventors sequenced the FAK genome and coding regions of 91 non-small cell lung cancer patients, and found four types of FAK variants in the cancer tissue of 7 non-small cell lung cancer patients: internal tandem duplication mutant (FAK-ITD), A1004S point mutation, deletion of exons Nos. 5-27, and expression of splicing variant FAK^{6.7}. These variants have not been reported in small cell lung cancer.

Currently, some small-molecule inhibitors of FAK are in early-stage clinical trials, and show good clinical prospects. Small-molecule inhibitors of FAK are mainly divided into two categories: ATP-competitive kinase inhibitors, which can inhibit the kinase activity of FAK; and structural inhibitors targeting the FAK molecular scaffold. Inhibitors targeting the FAK molecular scaffold are still in the early stage of research and development, while small-molecule kinase inhibitors of FAK have drawn more attention, and some of them are already in preclinical or clinical phase I/II trials. However, unlike classical lung cancer therapeutic targets such as EGFR and ALK, splicing variants of FAK in SCLC have not been studied.

### SUMMARY

One objective of the present disclosure is to provide a marker for clinical diagnosis of small cell lung cancer and a method for detecting the marker.

Another objective of the present disclosure is to provide novel targets for drug therapy of small cell lung cancer.

The present inventors have found in research that there are several splicing variants of FAK in SCLC cell lines and in SCLC patients' tissue, including FAK^{6.7}, FAK⁶ and FAK⁷. We further verified the expression of FAK^{6.7}, FAK⁶ and FAK⁷ in paraffin wax samples from 150 SCLC cases by RNA in situ hybridization. FAK is a non-receptor tyrosine kinase which is highly conserved over species, and phosphorylation of FAK (Tyr397) plays an important role in its functioning. The present inventors examined the expression level of p-FAK in FAK^{WT}, FAK^{6.7}, FAK⁶ and FAK⁷ by immunohistochemistry, and found that the presence of splicing variants of FAK can significantly enhance the phosphorylation of FAK (Tyr397). Some small-molecule inhibitors of FAK have entered early clinical trials and showed good clinical prospects. It was found that FAK-targeting drugs in clinical trials showed better inhibitory effects on SCLC with various types of FAK splicing variants. The present inventors identified SCLC-specific FAK splicing variants, which have great clinical significance in clinical diagnosis of SCLC, targeted therapy of SCLC, and guiding the clinical applications of FAK inhibitors.

Therefore, in one aspect, an embodiment of the present disclosure provides use of FAK splicing variants as a target in clinical diagnosis of SCLC, screening and/or manufacture of a medicament for treating SCLC.

Specifically, an embodiment of the present disclosure provides use of reagents for detecting FAK splicing variants in the manufacture of diagnostic agents for diagnosis of SCLC.

According to specific embodiments of the present disclosure, focal adhesion kinase splicing variants can be used alone as a marker for clinical diagnosis of SCLC, or used in combination with other clinical diagnostic markers for clinical diagnosis of SCLC.

An embodiment of the present disclosure also provides use of focal adhesion kinase splicing variants as a target in the screening and/or manufacture of a medicament for treating SCLC.

An embodiment of the present disclosure also provides use of substances targeting FAK in the manufacture of a medicament for treating SCLC.

According to specific embodiments of the present disclosure, the substances targeting FAK comprise agents that inhibit FAK and/or inhibit the kinase activity of FAK.

According to specific embodiments of the present disclosure, in the use of substances targeting FAK in the manufacture of a medicament for treating SCLC, the SCLC may be SCLC having the wild-type FAK splicing form and/or FAK splicing variants. The substances targeting FAK according to the present disclosure can be used to manufacture a medicament for treatment of SCLC having various types of FAK splicing variants (wild type, FAK^{6.7}, FAK⁶, FAK⁷).

According to specific embodiments of the present disclosure, the FAK splicing variants according to the present disclosure comprise one or more of FAK^{del33}, FAK^{del26}, FAK^{+6.7} (box6,7, PWR) and FAK^{6.7} (box6,7), FAK⁶ (box6), FAK⁷ (box7), FAK^{6.28} (box6,28).

In another aspect, embodiments of the present disclosure also provide use of inhibitors that inhibit FAK and/or the phosphorylation activity of FAK in the manufacture of a medicament for treatment of tumors. The inhibitors may be for example small-molecule drugs, including but not limited to PF-562271, VS-6063, BI-3663, P12PF inhibitors, and the like.

According to specific embodiments of the present disclosure, the tumor (small cell lung cancer) includes cancer sensitive and/or resistant to immunotherapy.

In another aspect, embodiments of the present disclosure also provide a method for detecting FAK splicing variants, which is used in clinical diagnosis of SCLC.

According to specific embodiments of the present disclosure, detecting the level of FAK splicing variants comprises detecting the level of gene expression of FAK splicing variants, or detecting the level of expression of p-FAK protein in FAK splicing variants. The detection may be performed by any suitable methods known in the art, including, for example, but not limited to, methods that utilize immunohistochemistry, RNA in situ hybridization, or Western blot. Reagents for detecting FAK splicing variants include, but are not limited to, detection reagents used in these detection methods. In some embodiments of the present disclosure, the reagents for detecting FAK splicing variants include: reagents for detecting the level of phosphorylation of FAK (Tyr397) by immunohistochemistry, and/or reagents for detecting FAK splicing variants by RNA in situ hybridization.

According to specific embodiments of the present disclosure, treatment of a tumor (small cell lung cancer) comprises treating the tumor by inhibiting FAK and/or FAK phosphorylation activity, or treating the tumor by combination use of inhibiting FAK and/or FAK phosphorylation activity with an immunotherapy.

In some embodiments, the tumor (small cell lung cancer) includes cancer sensitive and/or resistant to immunotherapy.

In yet another aspect, embodiments of the present disclosure also provide a pharmaceutical composition for treatment of a tumor, comprising an agent that inhibits FAK and/or FAK phosphorylation activity, and optionally an immune checkpoint antibody. The immune checkpoint may, for example, include one or more of PD-1, PD-L1, and CTLA-4. In some specific embodiments, the immune checkpoint antibody is preferably a PD-1 and/or PD-L1 antibody, thus a novel approach to immunotherapy of cancer is provided.

In specific embodiments of the present disclosure, the present inventors experimentally examined SCLC cell lines and 150 samples from SCLC patients by RNA in situ hybridization, and 35 cases were validated by RT-PCR, Sanger sequencing of PCR products, and RNA in situ hybridization. Various FAK splicing variants were found in as high as 83% of the SCLC cell lines and the patient samples, providing support for the use as a clinical diagnostic marker for SCLC. Overexpression of FAK splicing variants in SCLC cell lines significantly enhances cell proliferation, migration, and kinase activity. The results of treatment of the cell lines with different FAK-targeting clinical drugs, MTT, and western blot showed that cell lines containing FAK splicing variants had greater sensitivity to these drugs (lower IC₅₀ and phosphorylation of FAK (Tyr397) as compared to wild-type cells). The results of tumor-bearing mouse model also showed that tumors with FAK splicing variants had higher drug sensitivity, providing evidence that FAK splicing variants can be used as a new target for treatment of SCLC.

### BRIEF DESCRIPTION OF DRAWINGS

FIGS. 1A to 1D display the experimental results of expression of FAK splicing variants in SCLC cell lines. FIG. 1A: gel electrophoresis of PCR products amplified with FAK^{6/7}-specific PCR primers from the cDNA reverse-transcribed from the RNA extracted from SCLC cell lines. FIG. 1B: comparison analysis obtained using Snapgene software from Sanger sequencing results for PCR products obtained with FAK^{6/7}-specific sequencing primers. FIG. 1C, overall PSI results of BOX6 and BOX7 in all cell lines. FIG. 1D, PSI results of BOX6 and BOX7 in each cell line.

FIGS. 2A to 2D show the experimental results of expression of FAK splicing variants in SCLC patients' tissue. FIG. 2A: gel electrophoresis of PCR products amplified with FAK^{6/7}-specific PCR primers from the cDNA reverse-transcribed from the RNA extracted from SCLC patients' tissue. FIG. 2B, paraffin sections (4 μM) of SCLC patient tissue stained by RNA in situ hybridization (left) and immunohistochemistry (right) to detect the levels of FAK splicing variants and FAK phosphorylation in the patients' tissue. FIG. 2C, Western blot analysis of the cancer tissue from samples of 8 cases of lung cancer patient using anti-FAK, p-FAK, YAP and Actin antibodies, showing the expression levels of the 8 samples which are marked with the patient numbers. FIG. 2D, statistical result of FAK splicing variants in the tissue of SCLC patients.

FIGS. 3A to 3D show that overexpression of various FAK splicing variants significantly promoted proliferation, migration and kinase activity in cells. FIG. 3A, Western blot of DMS114 (FAK wild-type cells), in which FAK splicing variants are overexpressed. FIG. 3B, FAK splicing variants are overexpressed in DMS114 (FAK wild-type cells), and Incucyte assay showed that FAK⁷ and FAK^{6.7} significantly promoted cell proliferation. FIG. 3C, DMS114 (FAK wild-type cells) overexpressed FAK splicing variants, and Transwell assay showed that FAK⁷ and FAK^{6.7} significantly promoted cell migration. FIG. 3D, DMS114 (FAK wild-type cells) overexpressed FAK splicing variants, and the kinase activity assay showed that FAK⁷ and FAK^{6.7} significantly enhanced the kinase activity in the cells.

FIGS. 4A to 4C show the sensitivity of SCLC cell lines with different FAK splicing variants to the FAK inhibitor PF-562271. FIG. 4A shows cell viability, measured by CCK8, of SCLC cell lines having different FAK splicing variants after 48 hours treatment with different concentrations of FAK inhibitor PF-562271. FIG. 4B, Western blot analysis performed with anti-p-FAK and FAK antibodies on cell precipitates of FAK^{WT} cell line LCO217 that was treated with different concentrations of FAK inhibitor PF-562271 for 48 h. FIG. 4C, Western blot analysis performed with anti-p-FAK and FAK antibodies on cell precipitates of FAK^{6.7} cell line H82 that was treated with different concentrations of FAK inhibitor PF-562271 for 48 h.

FIGS. 5A to 5D show the sensitivity of SCLC cell lines with different FAK splicing variants to the FAK inhibitor PF-562271 in another experiment. FIG. 5A shows cell viability, measured by CCK8, of SCLC cell lines having different FAK splicing variants after 48 hours treatment with different concentrations of FAK inhibitor PF-562271. FIG. 5B, Western blot analysis performed with anti-p-FAK and FAK antibodies on cell precipitates of FAK^{WT} cell line H1339 that was treated with different concentrations of FAK inhibitor PF-562271 for 48 h. FIG. 5C, Western blot analysis performed with anti-p-FAK and FAK antibodies on cell precipitates of FAK^{6.7} cell line H82 that was treated with different concentrations of FAK inhibitor PF-562271 for 48 h. FIG. 5D, cell cycle changes, measured by flow cytometry, of FAK^{WT} cell line H1339 and FAK^{6.7} cell line H82 that were treated with different concentrations of FAK inhibitor PF-562271 for 24 hours.

FIGS. 6A to 6B show the sensitivity of SCLC tumor-bearing models having different FAK splicing variants to the FAK inhibitor PF-562271. FIG. 6A shows the sensitivity of DMS114 (FAK wild-type cells) to the inhibitor PF-562271. FIG. 6B shows the sensitivity of H82 (FAK^{6.7} cells) to the inhibitor PF-562271.

### DETAILED DESCRIPTION

The embodiments of the present disclosure and the beneficial effects produced thereby are described below in detail by specific examples, which are intended to help the readers have a better understanding of the spirit and features of the present disclosure, and are not intended to limit the implementable scope of the present disclosure. In the examples, the experimental methods for which no specific conditions are indicated are conventional methods with conventional conditions well known in the art, or are operated in accordance with the conditions recommended by the instrument manufacturers.

### Example 1

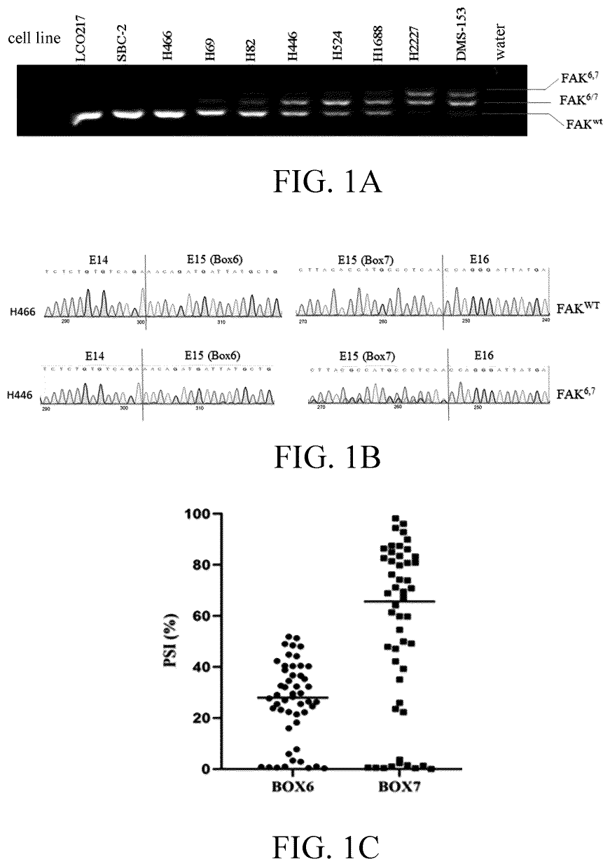

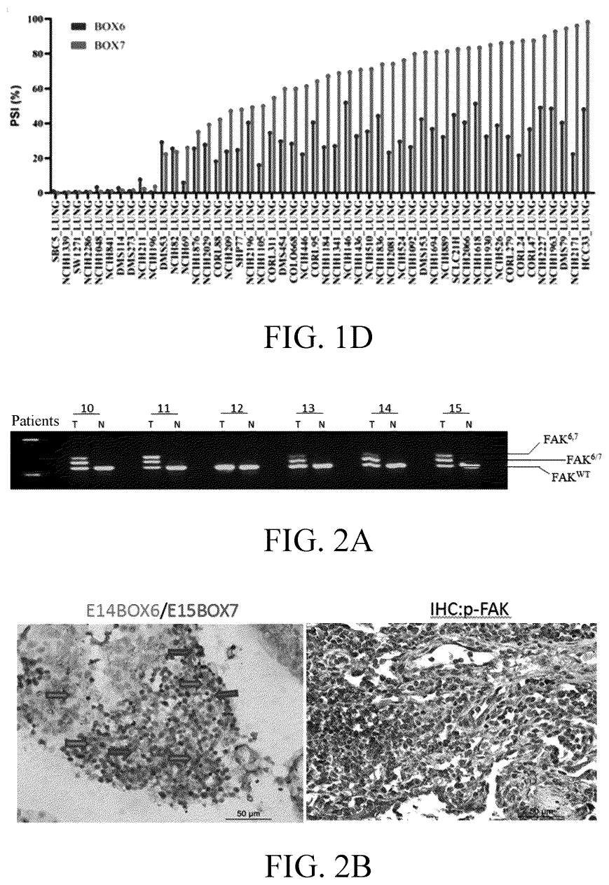

In this example, SCLC cell lines were cultured, including LCO217 (PDC cells of SCLC patients); SBC-2 (purchased from BeNa Culture Collection); H1339, H69, H82, H446, H2227, DMS153 (purchased from the National Infrastructure of Cell Line Resource); H524 (Kunming Cell Bank, Chinese Academy of Sciences); H1688 (Shanghai Institutes for Biological Sciences, Chinese Academy of Sciences). RNA was extracted by the Trizol method from the SCLC cell lines, reverse-transcribed into cDNA, and PCR-amplified with FAK^{6/7}-specific PCR primers. The PCR products were identified by gel electrophoresis, with the results shown in FIG. 1A. The cDNA of SCLC cell lines was PCR-amplified with FAK^{6/7}-specific sequencing primers, the PCR products were sequenced according to Sanger, and the sequences were compared and analyzed by Snapgene software, with the results shown in FIG. 1B.

The reference file containing the cell line exon ratio data (CCLE_RNAseq_ExonUsageRatio_20180929.gct.gz) and cell line annotation information file (Cell_lines_annotations_20181226.txt) were downloaded from the CCLE database (https://portals.broadinstitute.org/ccle/data). All exon, including ratio information, of the PTK2 gene (gene_id: ENSG00000169398.15) was retrieved from it, and further analyzed to obtain the exon ratios comprised in BOX6 and BOX7, also known as Percent Spliced Index (PSI). Each cell line can have two PSI values for exons since the sequencing starts from both ends, so the average of both values was taken as the final PSI value. The PSI values for the type of SCLC cell lines (tcga_code: SCLC) were then selected with the assistance of cell line annotation information. The overall PSI results for BOX6 and BOX7 for all cell lines are shown in FIG. 1C, and the PSI results for BOX6 and BOX7 for each cell line are shown in FIG. 1D. The position information of BOX6 and BOX7 is as follows: BOX6 (exon:chr8_141779691_141779674_PTK2_3p, chr8_141779691_141779674_PTK2_5p) BOX7 (exon:chr8_141772487_141772467_PTK2_3p, chr8_141772487_141772467_PTK2_5p).

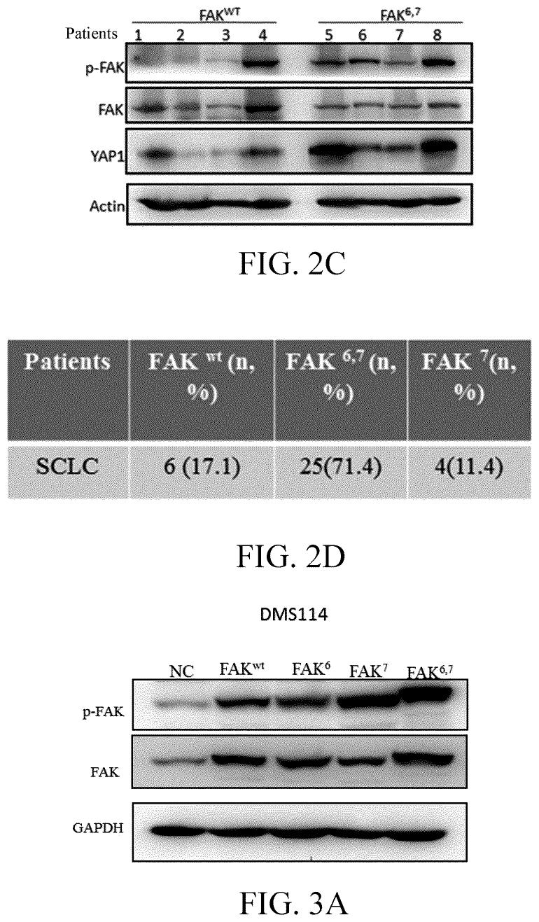

In order to further verify the in vivo level of FAK splicing variants in the tissue of SCLC patients, RNA was extracted from the tissue of SCLC patients, reverse-transcribed into cDNA, and PCR-amplified with FAK^{6/7}-specific PCR primers, and the PCR products were identified by gel electrophoresis, with the results shown in FIG. 2A. As shown in FIG. 2B, paraffin sections (4 μM) of the tissue of SCLC patients were stained by RNA in situ hybridization (left) and by immunohistochemistry (right) to detect the FAK splicing variants and FAK phosphorylation levels in the patients' tissue. Then a western blot analysis was performed on the cancer tissue from samples of 8 cases of lung cancer patient using anti-FAK, p-FAK, YAP and Actin antibodies. The expression levels of the 8 samples which are marked with the patient numbers are shown in FIG. 2C. Statistical analysis was performed on the FAK splicing variants in the tissue of SCLC patients, as shown in FIG. 2D.

### Example 2

In this Example, the role of the expression of FAK splicing variants in the clinical diagnosis of SCLC was investigated. Lung cancer samples of SCLC were taken and the expression level of FAK splicing variants was detected by RNA in situ hybridization. The detailed detection method was as follows:
1) Dewaxing: the samples were placed in a 72° ° C. oven to be preheated for 2.5 h, and then put in fresh xylene I&II&III for 10 minutes each;
2) Hydration: the samples were put in anhydrous ethanol I&II for 2 minutes each, air-dried at room temperature, and used within 24 h or used directly in the next step;
3) Hydrogen peroxide treatment: 5-8 drops of hydrogen peroxide were added to the slide, kept for 10 min at room temperature, and the slide was rinsed in ddH₂O 3-5 times;
4) Repair: 1× repair solution was boiled at 100° ° C. or higher, the slides were slowly put in the repair solution, covered with an aluminum foil and repaired for 15 min; immediately after the repairing, the slides were put into ddH₂O immediately and rinsed 3-5 times; then put in fresh anhydrous ethanol, washed 3-5 times and then air-dried thoroughly;
5) Protease treatment: a water-blocking circle was drawn around the tissue with a histochemical pen, 5 drops of protease IV was added onto the slide tissue, which was covered with a plastic film, put in a hybridization oven)(40° ° C. for 30 min, and then rinsed in ddH₂O 3-5 times;
6) probes were placed in a 40° ° C. water bath for 10 min, and then cooled to room temperature;
7) excess liquid was removed from the slides by gentle tapping, about 4 drops of suitable probes were added to completely cover the whole tissue, which was put in a wet box and placed in a hybridization oven at 40° C. for 2 h;
8) the slide after hybridization was removed and washed with a wash buffer 2 times, for 2 min each;
9) Amp1 to Amp8 hybridization treatment; excess liquid was removed from the slides by gentle tapping, Amp1 was added to completely cover the whole tissue, which was treated at 40° ° C. for 30 min; then excess liquid was removed, and the slides were washed with a wash buffer 2 times, for 2 min each time; then the same operation was performed with Amp2 (treated at 40° C. for 30 min); Amp3 (treated at 40° C. for 15 min); Amp4 (treated at 40° ° C. for 30 min); Amp5 (treated at 40° C. for 30 min); Amp6 (treated at 40° ° C. for 15 min); Amp7 (treated at room temperature for 30 min); and Amp8 (treated at room temperature for 15 min);
10) Detection of red signal:
(i) Fast Red-B was rapidly centrifuged, making sure the liquid was at the bottom of the tube, with 120 μl per slice;
(ii) B:A=1:60, 2 μl B was added to 120 μl A, mixed thoroughly, and used within 5 min protected from light;
(iii) Excess liquid was removed, 120 μl RED working solution was added dropwise to cover the sample;
(iv) The sample was put in a wet box, covered with a lid, and left at room temperature for 10 min;
(v) The slide was removed, and washed with a wash buffer for 2 min at room temperature, which was repeated 3-5 times;
11) Amp9 to Amp12 hybridization treatment: excess liquid was removed from the slides by gentle tapping, Amp9 was added to completely cover the whole tissue, which was treated at 40° ° C. for 15 min; then excess liquid was removed, and the slides were washed with a wash buffer 2 times, for 2 min each time; then the same operation was performed with Amp10 (treated at 40° ° C. for 15 min); Amp11 (treated at room temperature for 30 min); and Amp12 (treated at room temperature for 15 min);
12) Detection of green signal:
(i) Fast Green-B was rapidly centrifuged, making sure the liquid was at the bottom of the tube, with 120 μl per slice;
(ii) B:A=1:50, 2.4 μl B was added to 120 μl A, mixed thoroughly, and used within 5 min protected from light;
(iii) Excess liquid was removed, 120 μl GREEN working solution was added dropwise to cover the sample;
(iv) The sample was put in a wet box, covered with a lid, and left at room temperature for 10 min;
(v) The slide was removed, and washed with a wash buffer for 5 min at room temperature;
(vi) The slide was rapidly rinsed with fresh distilled water within 30 s;
13) Counterstaining
(i) About 100 μl of 50% hematoxylin staining solution was added onto the slide to stain for 1 min at room temperature, and the tissue turned purple;
(ii) The slide was put in and out of a tank with flowing tap water 3-5 times until the slide was clean, and the tissue was purple;
(iii) The slide was put in and out of 0.02% ammonia 3-5 times, and the tissue turned blue;
(iv) The slide was put in and out of a tank with flowing tap water 3-5 times until the slide was clean, and the tissue was blue.
14) Mounting: The slide was placed in an oven at 60° C. for 15-30 min until the slide was completely dried, and cooled at room temperature for 5 min; after adding 1-2 drops of a mounting medium, the slide was coved carefully with a coverslip without air bubbles, and dried in a ventilated place.

The results are shown in FIGS. 2A to 2D.

FIGS. 2A to 2D show that the cancer tissue of SCLC patients had a very high level of expression of FAK splicing variants (FAK^{6.7}, 71.4%; FAK^{6/7}, 11.4%) and FAK phosphorylation (by immunohistochemistry), whereas there was no FAK splicing variant in the paracancerous tissue. These results indicate that detection of the expression level of FAK splicing variants in clinical patients with lung cancer can serve as an important tool in clinical diagnosis of SCLC.

### Example 3

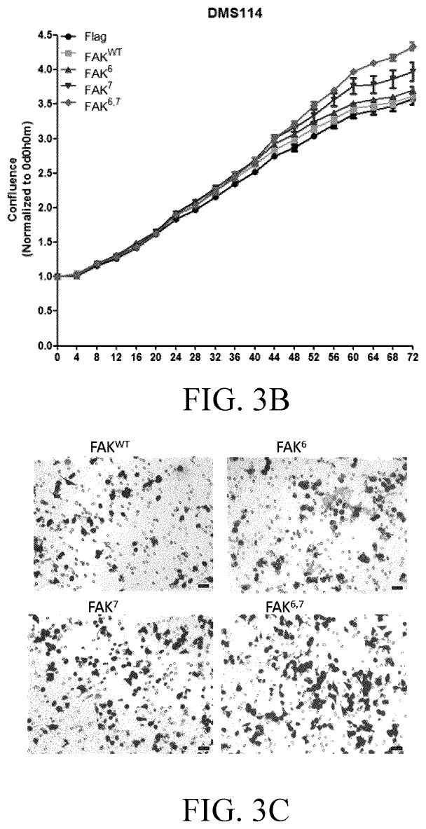

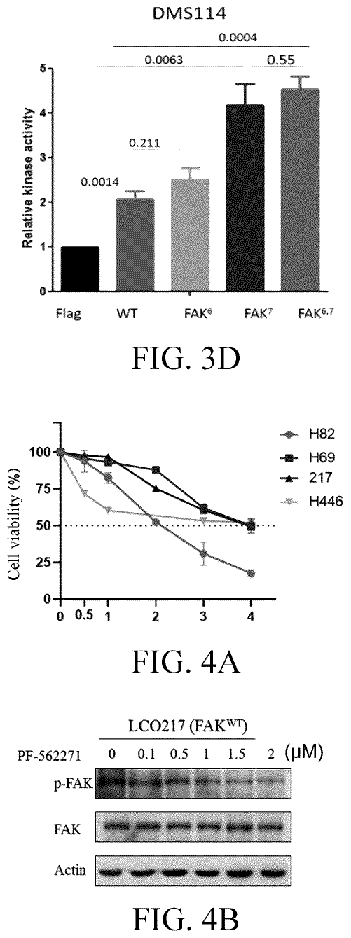

In order to further investigate the role of FAK splicing variants in SCLC, the present inventors constructed plasmids having different types of FAK splicing variants (FAK^{6.7}, FAK⁷, FAK⁶, FAK″, FAK^{flag}), and transfected DMS114:FAK^{WT} cells with them by Lipo3000, and the results of transfection were examined by Western blot, as shown in FIG. 3A. Incucyte assay showed the effect on cell proliferation, as shown in FIG. 3B. Transwell assay showed the effect on cell migration, as shown in FIG. 3C. Kinase activity assay showed the effect of different variants on the kinase activity, as shown in FIG. 3D. The results showed that overexpression of various FAK splicing variants significantly promoted cell proliferation, migration and kinase activity.

### Example 4

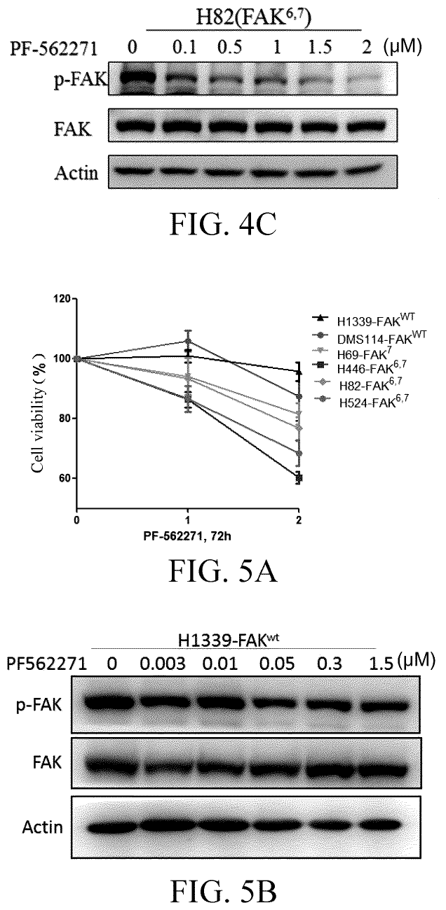

To further evaluate the role of FAK splicing variants in the treatment of SCLC, the present inventors treated SCLC cell lines having different FAK splicing variants (H82:FAK^{6.7}, H446:FAK^{6.7}, H69:FAK⁷, LCO217:FAK^{WT}) with different concentrations (μM) of the FAK inhibitor PF-562271 for 48 hours, and then measured the cell viability by CCK8, with the results shown in FIG. 4A.

A Western blot analysis was performed with anti-p-FAK and FAK antibodies on cell precipitates, obtained from FAK^{WT} cell line LCO217 at 48 h after treatment with different concentrations of FAK inhibitor PF-562271, with the results shown in FIG. 4B.

A Western blot analysis was performed with anti-p-FAK and FAK antibodies on cell precipitates, obtained from FAK^{6.7} cell line H82 at 48 h after treatment with different concentrations of FAK inhibitor PF-562271, with the results shown in FIG. 4C.

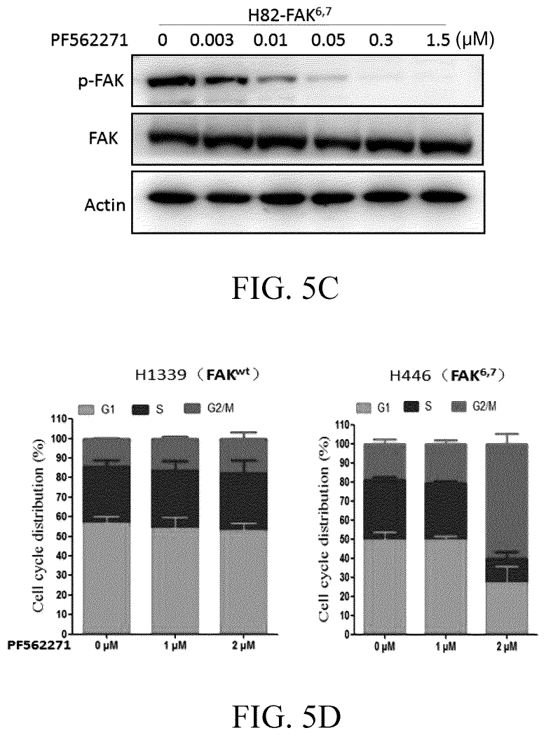

In another experiment, the present inventors treated SCLC cell lines having different FAK splicing variants (H524:FAK^{6.7}, H82:FAK^{6.7}, H446:FAK^{6.7}, H69:FAK⁷, H1339:FAK^{WT}, DMS114:FAK^{WT}) with different concentrations of the FAK inhibitor PF-562271 for 48 hours, and then measured the cell viability by CCK8, with the results shown in FIG. 5A. A Western blot analysis was performed with anti-p-FAK and FAK antibodies on cell precipitates of FAK^{WT} cell line H1339 that was treated with different concentrations of FAK inhibitor PF-562271 for 48 h, with the results shown in FIG. 5B. A Western blot analysis was performed with anti-p-FAK and FAK antibodies on cell precipitates of FAK^{6.7} cell line H82 that was treated with different concentrations of FAK inhibitor PF-562271 for 48 h, with the results shown in FIG. 5C. FAK^{WT} cell line H1339 and FAK^{6.7} cell line H82 that were treated with different concentrations of FAK inhibitor PF-562271 for 24 hours were measured by flow cytometry for cell cycle changes, with the results shown in FIG. 5D.

### Example 5

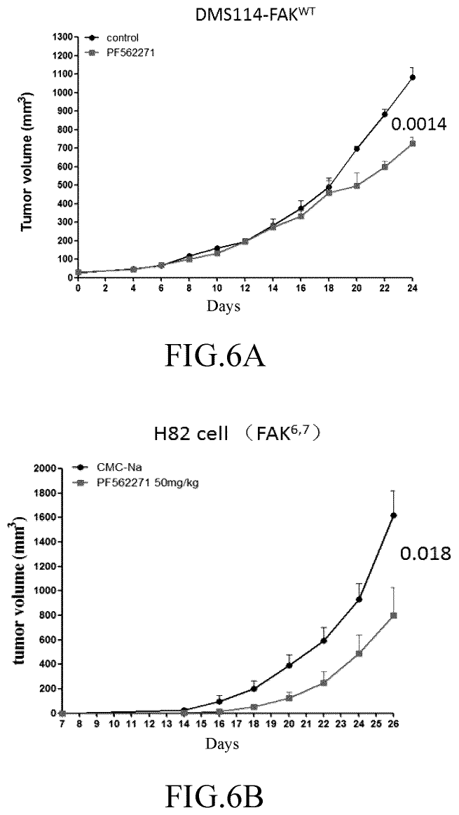

In order to evaluate the role of FAK splicing variants in treatment of SCLC in mice, the present inventors inoculated mice with DMS114:FAK^{WT} and H82:FAK^{6.7} cell lines subcutaneously to induce tumors, and intragastrically administered PF562271 at 50 mg/kg once a day, and the tumor volume of the mice was measured on every other day, with the results shown in FIGS. 6A and 6B.

Embodiments of the present disclosure have been describe above, and it should be noted that for a person of ordinary skill in the art, a number of improvements and modifications can be made without departing from the principles of the present disclosure, and these improvements and modifications should also be within the scope of protection of the present disclosure.

What is claimed is:

1. Use of reagents for detecting focal adhesion kinase (FAK) splicing variants in the manufacture of a diagnostic agent for diagnosis of small cell lung cancer (SCLC).

2. Use of FAK splicing variants as a target in the screening and/or manufacture of a medicament for treating SCLC.

3. The use according to claim 1 or 2, wherein the SCLC is SCLC having a wild type FAK and/or a FAK splicing variant.

4. The use according to any one of claims 1 to 3, wherein the FAK splicing variant comprises one or more of FAK^{del33}, FAK^{del26}, FAK^{+6,7} and FAK^{6,7}, FAK⁶, FAK⁷, FAK^{6.28}.

5. The use according to claim 4, wherein the FAK splicing variant is FAK^{6,7}.

6. The use according to claim 1 or 2, wherein the SCLC includes a cancer sensitive and/or resistant to immunotherapy.

7. The use according to claim 1, wherein the reagents for detecting FAK splicing variants comprise: a reagent for detecting the level of phosphorylation of FAK Tyr397 by immunohistochemistry, and a reagent for detecting FAK splicing variants by Western blotting and/or RNA in situ hybridization.
